(19) United States
(12) Patent Application Publication
(10) Pub. No.: US 2023/0337963 A1
(43) Pub. Date: Oct. 26, 2023

(54) OLFACTORY IMPAIRMENT TESTING AND TRAINING PLATFORM

(71) Applicant: SENSIFY, INC., Tarrytown, NY (US)

(72) Inventors: Subhanjan Mondal, Middleton, WI (US); Farooq Waheed, New York, NY (US); Rauf Waheed, New York, NY (US)

(73) Assignee: SENSIFY, INC., Tarrytown, NY (US)

(21) Appl. No.: 17/900,133

(22) Filed: Aug. 31, 2022

Related U.S. Application Data
(60) Provisional application No. 63/334,247, filed on Apr. 25, 2022.

(51) Int. Cl. A61B 5/00; G16H 50/20

(52) U.S. Cl. CPC A61B 5/4011; G16H 50/20; A61B 2560/0406

(57) ABSTRACT

An olfactory testing platform for testing for olfactory performance and for prompting olfactory training. The kit contains a plurality of inhaler sticks, each of which includes a first indicia that is computer readable, and a second indicia that is human readable. Each inhaler stick provides a scent that is different than every other inhaler stick in smell or concentration. A computer application running on a smart device having a camera is used to identify each inhaler stick in the kit using the first indicia and to prompt a user to select a detected scent from a plurality of options in response to presentation of the first indicia to the camera of the smart device. The smart devise also captures cognitive functions and integrates performance of various cognitive domains with olfactory performance.

### CROSS-REFERENCE TO RELATED APPLICATIONS

The present application claims priority to U.S. Provisional Application No. 63/334,247, filed on Apr. 25, 2022.

### BACKGROUND OF THE INVENTION

### 1. Field of the Invention

The present invention relates to medical diagnostics tests and, more particularly, to an olfactory impairment testing kit and approach.

### 2. Description of the Related Art

Reversible and irreversible olfactory impairment is indicative of multiple physiological or pathological conditions. For example, there are many causes for reversible olfactory impairment including upper respiratory infections, nasal polyps, medications such as antibiotics and blood pressure pills, and mild cognitive impairment. Irreversible olfactory impairment may arise as a consequence of natural aging or pathological conditions, such as dementia and Alzheimer’s disease. Cognitive impairment and olfactory loss are also associated with several autoimmune diseases like rheumatoid arthritis, Sjögren syndrome, multiple sclerosis, and systemic lupus erythematosus. Olfactory function testing has thus been used for assessment of mild cognitive impairment, dementia, Parkinson’s disease and Alzheimer’s disease.

Conventional olfactory testing requires the involvement of a healthcare professional to administer the test and multiple hospital visits, which increases the cost of screening for early assessment of conditions such as cognitive impairment. As a result, olfactory testing is often not used, which can lead to delay in disease diagnosis and the need for more tests such as brain scans.

Accordingly, there is a need in the art for an olfactory testing approach that is less expensive and integration with cognitive self-assessments that can be made available for general use, thereby improving the early detection and diagnosis of cognitive impairments. Early detection and diagnosis of these conditions can provide an opportunity for appropriate treatment and lifestyle changes, such as proper diet, exercise, stress, and stress reduction, that can help restore normal cognition or delay progression of cognitive loss as well as help in development of improved therapies for cognitive impairment. In addition, smell retraining techniques can be used for re-training of the olfactory system after a period of loss of olfactory sensation, such as that associated with viral infections and, more specifically, SARS-CoV-2.

### BRIEF SUMMARY OF THE INVENTION

The present invention is a patient directed olfactory testing platform that can be used by individuals to test for reversible and irreversible olfactory loss and to perform olfactory training to help restore a lost sense of smell. The system also relates to a system that integrates olfactory performance with various cognitive domains. More specifically, the present invention comprises an olfactory testing and training platform having a kit with a plurality of scents and an accompanying computer application that can allow an individual to perform olfactory testing and training in a randomized manner and, optionally, perform preliminary diagnosis or screening analyses. The kit contains a plurality of inhaler sticks, each of which includes a first indicia that is computer readable, and a second indicia that is human readable, wherein each of the inhaler sticks provides a scent that is different than every other inhaler stick or similar with varied intensities. The scents can be similar in odor arising from a single chemical agent or a combination of multiple chemical entities (e.g. phenyl ethyl alcohol and rose essence, both smell of rose, one is a single chemical compound, other is a mixture of chemicals, but both generating a common familiar scent). A smart device having a camera and a computer application programmed to identify each inhaler stick in the kit by the first indicia is used to provide the user with a prompt to select a detected scent for a given inhaler stick from a plurality of options in response to presentation of the first indicia of the given inhaler stick to the camera. This method is used to assess odor identification score. The second indica is used to assess odor discrimination and odor intensity/ threshold.

In a first embodiment, the invention may be an olfactory testing platform comprised of a kit containing a plurality of scented objects such as inhaler sticks, each of which includes a first indicia that is computer readable, and a second indicia that is human readable, wherein each of the inhaler sticks provides a scent that is different than at least one other inhaler stick. The invention also includes a computer application configured to run on a smart device having a camera, where the computer application is programmed to identify each inhaler stick in the kit using the first indicia and to prompt a user to select a detected scent from a plurality of options in response to presentation of the first indicia to the camera of the smart device. The first indicia and the second indicia may be positioned on opposing sides of each of the plurality of inhaler sticks. The kit may include a rack having a plurality of receptacles for receiving the plurality of inhaler sticks and a cover that can be positioned in covering relation to the rack to enclose the plurality of inhaler sticks. At least one of the rack and the cover includes a third indicia containing information about the plurality of inhaler sticks. The computer application may be programmed to prompt a user to scan each inhaler stick in the kit using the first indicia. The computer application may be programmed to prompt a user to select more than one of the plurality of inhaler sticks and identify the scent of each of the more than one of the plurality of inhaler sticks. The computer application may be programmed to prompt a user to select at least two of the plurality of inhaler sticks and to discriminate between the at least two of the plurality of inhaler sticks according to a strength of the scent associated with each of the at least two of the plurality of sticks. The computer application may be programmed to prompt a user to scan one of the plurality of inhaler sticks and to display the scent associated with that inhaler stick. The computer application may be programmed to display a digit corresponding to one of a series of symbols in a table and to allow the user to draw the one of the series of symbols corresponding to the digit. The computer application may be programmed to record the eye movements of the user when the user draws the one of the series of symbols corresponding to the digit.

In another embodiment, the invention may be a method of performing olfactory testing. The method involves providing a plurality of scented objects such as inhaler sticks, each of which includes a first indicia that is computer readable, and a second indicia that is human readable, wherein each of the inhaler sticks provides a scent that is different than at least one other inhaler stick and then using a computer application running on a smart device having a camera to identify each inhaler stick in the kit upon presentation of the first indicia to the camera and to prompt a user to select a detected scent from a plurality of options after presentation of the first indicia to the camera. The method may also involve the step of using the computer application to prompt a user to scan each inhaler stick in the kit using the first indicia. The method may also involve the step of using the computer application is programmed to prompt a user to select more than one of the plurality of inhaler sticks and identify the scent of each of the more than one of the plurality of inhaler sticks. The method may also involve step of using the computer application to prompt a user to select at least two of the plurality of inhaler sticks and to discriminate between the at least two of the plurality of inhaler sticks according to a strength of the scent associated with each of the at least two of the plurality of sticks. The method may also involve the step of using the computer application to prompt a user to scan one of the plurality of inhaler sticks and to display the scent associated with that inhaler stick. The method may also involve the step of using the computer application to display a digit corresponding to one of a series of symbols in a table and to allow the user to draw the one of the series of symbols corresponding to the digit. The method may also involve the step of using the computer application to record the eye movements of the user when the user draws the one of the series of symbols corresponding to the digit.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of an olfactory testing and training platform according to the present invention.

FIG. 2 is a perspective view of an inhaler stick for an olfactory testing and training platform as it is rotated.

FIG. 3 is an exploded view of an inhaler stick for an olfactory testing and training platform according to the present invention.

FIG. 4 is a top view of a kit for enclosing a plurality of inhaler sticks according to the present invention.

FIG. 5 is a flowchart of a first testing procedure using a platform according to the present invention.

FIG. 6 is a flowchart of a second testing procedure using a platform according to the present invention.

FIG. 7 is a flowchart of a third testing procedure using a platform according to the present invention.

FIG. 8 is a flowchart of a fourth testing procedure using a platform according to the present invention.

FIG. 9 is a flowchart of a first training procedure using a platform according to the present invention.

FIG. 10 is a schematic of an application for performing a digit symbol substitution test on a mobile device according to the present invention.

FIG. 11 is a schematic of an application for performing a digit symbol substitution test on a mobile device according to the present invention showing drawing of a symbol according to a user prompt.

FIG. 12 is a schematic of an application for performing a cognitive impairment diagnostic where a user is prompted to draw symbol including a circle.

### DETAILED DESCRIPTION OF THE INVENTION

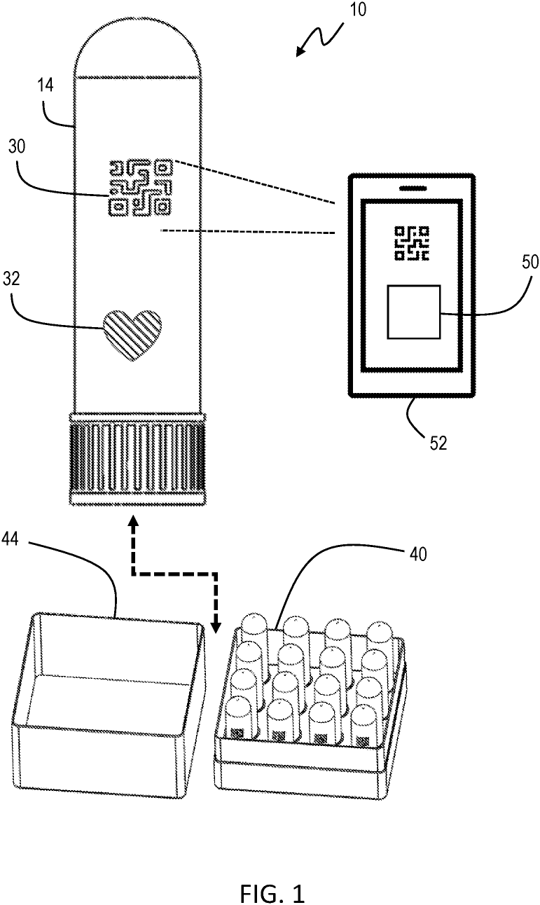

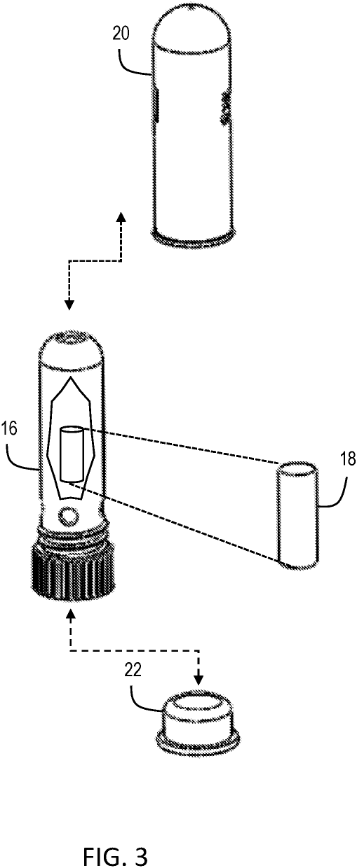

Referring to the figures, wherein like numerals refer to like parts throughout, there is seen in FIG. 1, a schematic of an olfactory platform 10 for olfactory testing and training. Platform 10 includes a kit 12 containing a series of scented objects such as inhaler sticks 14 for providing individual scents. As detailed in FIG. 3, each inhaler stick 14 comprises a wick holder 16 enclosing an absorbent wick 18 saturated with a particular scent. Inhaler stick 14 may be housed within a tubular outer cover 20 that is closed at one end and include a removeable lid 22 at the other end so that inhaler stick 14 can be preserved when not in use. As should be recognized by those of skill in the art, a user can remove lid 22 from outer cover 20 to access and withdraw stick 14 for use, and then replace stick 14 in cover 20 and seal cover 20 with lid 22, such as by press fitting lid 22 into the end of cover 20 to preserve stick 14 for future use. As seen in FIG. 3, kit 12 may include as many inhaler sticks 14 as desired or needed for the particular olfactory tests to be performed. For example, kit 12 may preferably include sixteen inhaler sticks 14, but could include as few as four, or more than sixteen as desired. As described below, inhaler sticks 14 could comprise any scented object that provides the desired scents and includes the identifying indicia for use with platform 10.

Wick 18 may be permeated to provide the ten basic smells that can be used for olfactory testing. These smells are fruity (e.g. mango, peach, melon), citrous/ lemon (e.g. lemon or oranges), floral (rose, chamomile). Mint/peppermint (e.g. menthol, peppermint, eucalyptus and camphor), sweet (e.g. chocolate, vanilla, caramel, and malty scents), toasted/nutty (e.g. peanut butter, popcorn, and almonds), woody/resinous (e.g. pine, fresh cut grass, musty, moldy, heavy, burnt, and smoky), chemical (synthetic smells recognized as danger signals, e.g. alcohols, disinfectants, ammonia, bleach, gasoline, and paint, leather), pungent (e.g. blue cheese, cigarette smoke, fecal matter/manure, sweat, onion, garlic), and putrid (e.g. rotting meat, sewage, burnt rubber, sulphuric acid, and household gas.

The smells provided by wicks 18 can be produced through natural sources, such as essential oils and odor producing chemicals that are generally recognized as safe (GRAS) such as those used for food additives. Essential oils for producing certain smells may include, without limitation, one of more of the following: cinnamon, turpentine, lemon, smoke, ammonia, chocolate, rose, paint thinner, banana, pineapple, gasoline, soap, cloves, orange, coffee, peppermint, peach, cherry chamomile, eucalyptus, wintergreen, thyme, sage, rosemary, garlic, and onion. Other substances such as fish oil or single compound odors including, without limitation, isoamyl acetate (for banana), phenyl ethyl alcohol (for rose), citronellal (for lemon), eugenol (clove), and eucalyptol (for eucalyptus) may be used. Mixtures of odorants may also be used. The essential oils, chemicals or mixtures, are used to wet absorbent wick 18, or may be diluted in vegetable oil or mineral oil as needed, and then applied to absorbent wick 18. Some inhaler stick/s may be wetted with only mineral oil or vegetable oil (no added smells). Some inhaler stick may not be wetted with any smell or odorant at all. Such inhaler sticks will be used if the user identifies the inhaler stick as having low or no smell tone.

Odorants in inhaler sticks 14 can be all unique smells, or some smells in the kit could be repeated odorants at the same or different concentration for use as an assessment of odor intensities and odor discrimination. Inhaler sticks 14 may also be designed to test for the ability to distinguish one smell from two similar smells. Such inhaler sticks 14 may also be used to identify the strongest or the weakest concentration among the options offered in the question.

Referring to FIG. 1, each inhaler stick 14 (and optionally also the corresponding wick holder 16) is labelled two forms of indicia. First, each inhaler stick 14 includes a first indicia 30 containing is labelled with an unique machine readable label, such as a QR code, that is not easily or practically decipherable by a user. Second, each inhaler stick 14 includes a second indicia 32 that is a unique symbol that is easily readable or recognizable by a user, e.g., a color coded shapes, alphanumeric codes, etc. As explained in detail below, first indicia 30 is used for interfacing with a computer application, such as by scanning with a mobile device having a camera that is running the application. Second indicia 32 allows a user to easily and quickly identify each inhaler stick 14 in response to prompting from the computer application. As should be understood, the use of first indicia 30 and second indicia 32 allow the computer application to identify each inhaler stick 14, including its specific characteristics, and guide a user using second indicia 32 that does not reveal the specific characteristics, so that procedures are conducted blindly to the user. For example, unique shapes and colors may be used for second indicia 32 during odor discrimination testing and odor intensity identification testing. Since each inhaler stick 14 is labelled with a unique shape-color symbol, it is difficult to memorize which symbol is associated with which smell, but it is a simple way to pick tubes out when called to do so by the software application, particularly when tests require a user to select more than one inhaler stick 14. The shapes may one or more of the following, including but not limited to, circle, triangle, square, start shape, heart shape, hexagon, octagon, polygon, oval/ellipse, crescent, parallelograms, semicircle, ring, trefoil, quatrefoil. The colors could be one of the following, including but not limited to green, red, yellow, orange, violet/ purple, blue, pink, black, white, brown. As noted above, inhaler sticks 14 could comprise any scented object that provides the desired scents and includes first indicia 30 and second indicia 32, including scented cards with impregnated scents that are readily released including in response to scratching by a user.

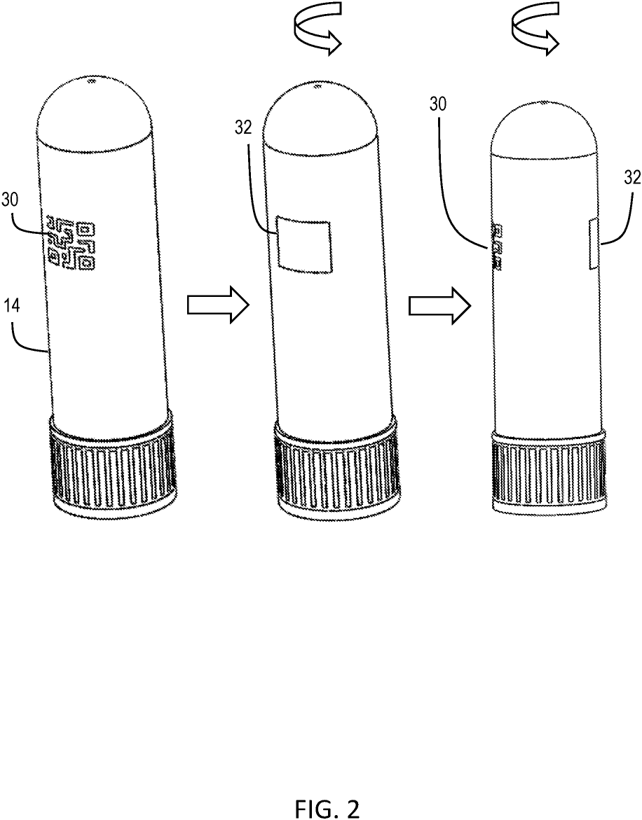

Referring to FIG. 2, first indica 30 and second indica 32 are in opposite sides of the inhaler stick 14, so that the user does not see the colored shape during scanning and answering of the inhaler stick 14. Machine readable indica 30 (QR code) contains information about the options to be presented for a particular smell and the correct answer for that smell. This information is used to create the self-administered test. In some instances, the odor presenting device can also be a scratch card containing a machine readable indica and a human readable indica as described above.

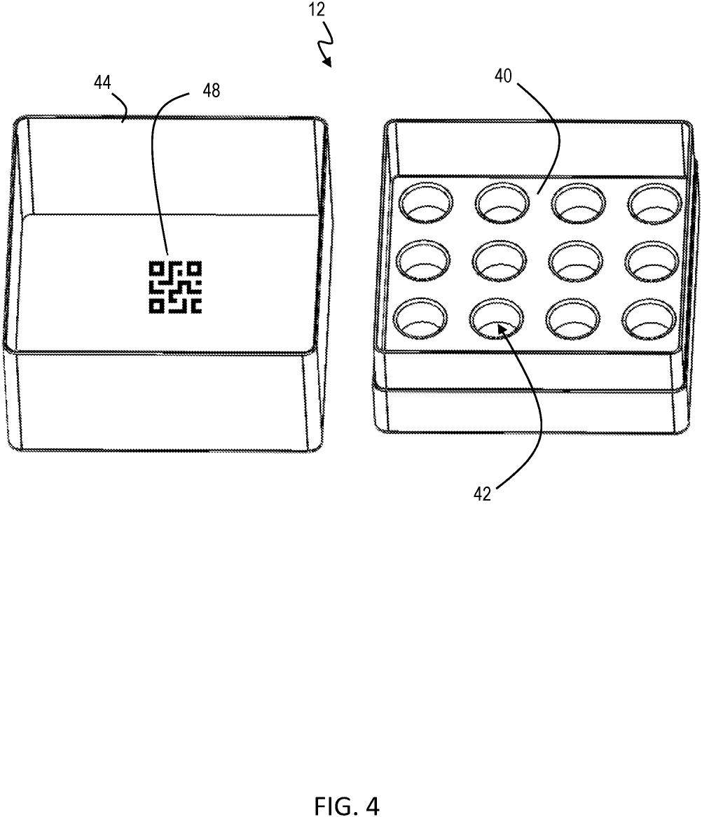

Referring to FIG. 4, inhaler sticks 14 may be housed in a foam rack 40 having a plurality of receptacles 42 dimensioned to receive sticks 14 and a cover 44 for enclosing the racked inhaler sticks 14. Cover 44 may be used to help support rack 40 for easy access to and visibility of inhaler sticks 14. For example, sixteen inhaler sticks 14 may be packaged in rack 40 with inhaler sticks 14 upright for easy visibility to the user. Rack 40 is preferably circular or square to help enable randomization and avoid any specific arrangements of inhaler sticks 14. Rack 40 may include a third indicia 48 for identifying the specific components therein as different kits with specifically curated inhaler sticks 14 for different testes or training can be in use.

Referring to FIG. 1, platform 10 further comprises a software application 50, such as a mobile device application, that is programmed according to the present invention to allow a user to perform self-directed tests and procedures. As is known in the art, a user of platform 10 may download a mobile application onto a smart device 52 having a camera for use in scanning first indicia 30, and optionally third indicia 48, and then prompting user for self-administering testing and training exercises. It should be recognized that software application 50 could also be implemented in the cloud provided that the user device has the ability to scan and recognize first indicia 30. As an example, software application 50 may be programmed to run a user questionnaire prior to any testing procedures to collect user data, such as name, age, gender, ethnicity, and location. Software application 50 may also asked a series of diagnostic background questions, such as whether the user has any current infection symptoms, positive viral tests, or cognitive impairment, whether the user is taking any medications (including which medications if applicable). Software application 50 then provides the user with prompts to perform a self-administered test or training exercise.

With respect to testing, four different types of odor tests may be self-administered: (1) odor identification that determines the ability to identify a particular odor when presented; (2) test-retest (intra-test consistency) that determines the ability to correctly identify the same smell the same way (same correctly or same smell incorrectly), more than one time. The intra-test consistency routine may be carried out by inducing a level of stress e.g. use of a large visible count-down, flashing light, buzzer or alike (3) odor discrimination that determines the ability to differentiate a particular odor from others; and (4) odor intensity determination that determined the level at which the ability to identify an odor functions.

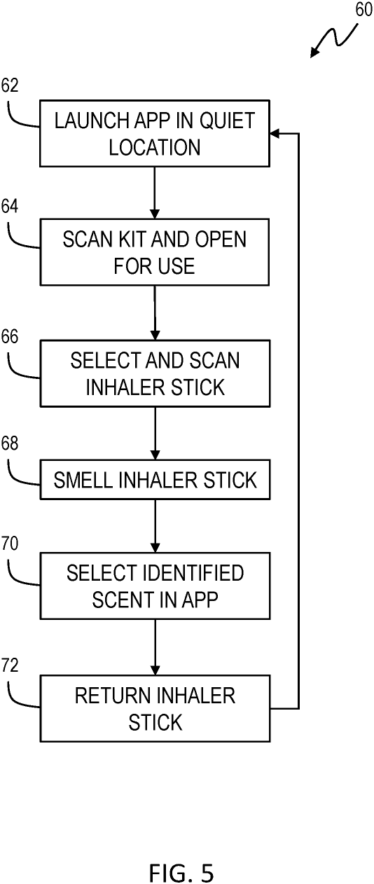

Referring to FIG. 5, an exemplary testing procedure 60 may begin with a user situated in a quiet place where the user can concentrate and avoid any distractions. In a first step 62, the user may then launch software application 50, such as by launching a corresponding mobile application from an Android or Apple mobile phone, and then in step 64 scan third indicia 48 of kit 12 to configure mobile application for the particular components of rack 40. The user may then open the rack 40 containing inhaler sticks 14 to allow for easy access to inhaler sticks 14. In a next step 66, a user may then select one inhaler stick 14 from rack 40 and scan first indicia 30. Software application 50 will prompt the user to smell inhaler stick 14 in step 68, and then in step 70 select the detected scent a list of options that the user believes is the detected scent of inhaler stick 14. The user may then place inhaler stick 14 in cover 44 and proceed to another inhaler stick 14 to repeat the identification process. This process is repeated until all inhaler sticks have been tested and placed in the cover 44.

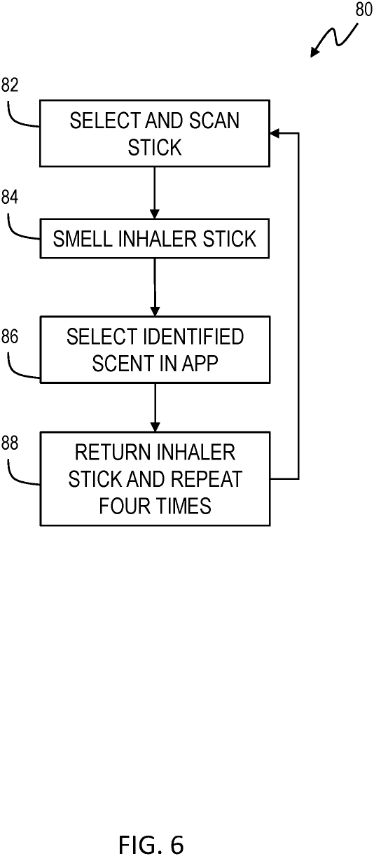

Referring to FIG. 6, after completing the initial identification described above, software application 50 may execute a retesting process 80 in step 82 by prompting the user to select a particular inhaler stick 14 from cover 44 and scan the inhaler stick 14. The user will then, in step 84, smell inhaler stick 14, and then select from the list of scents in step 86. Inhaler stick is then returned in step 88 back to the foam rack 40, and the entire process 80 repeated four times (or as many times as desired when configuring software application 50).

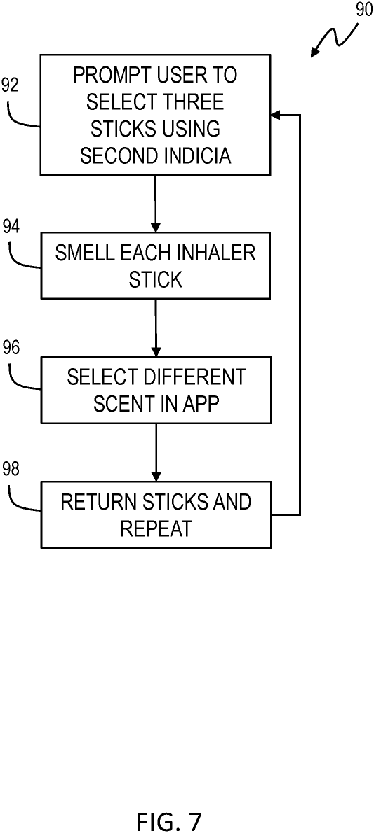

Referring to FIG. 7, software application 50 may also be programmed to perform an odor discrimination test 90 where the user is prompted in step 92 to pick three inhaler sticks 14 where software application 50 indicates which specific inhaler sticks 14 to pick by showing second indicia 34, such as the colored shapes discussed above. For this test, the user does not have to scan the QR code on inhaler sticks 14. In this test, the next step 94 is for the user to smell each of the three prompted inhaler sticks 14. Then, the user must identify in step 96 which one of the three tube is different from the other two and provide any other correct answers to questions as posed by software application 50. The three inhaler sticks 14 and then returned and the process repeated in step 98.

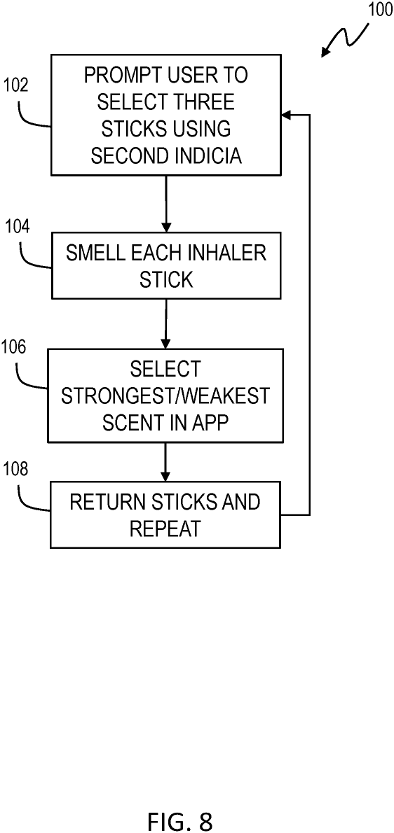

Referring to FIG. 8, software application 50 may further be programmed to execute an odor intensity discrimination test 100 where the user is prompted to select three inhaler sticks 14 using second indicia 34 in step 102, and then asked to identify which inhaler stick 14 has the strongest or weakest smell among the three inhaler sticks 14 in step 106. Inhaler sticks may be returned in step 108 and test 100 repeated.

Software application 50 is programmed to collect data during these tests, including a percentage of correct responses, test-retest reliability, average response time, odor discrimination score, and odor intensity discrimination score, smell test scan-to-scan response time as an indicator of executive function, odor specific response, olfactory intra-test consistency, olfactory response under stress. As certain odors arouse distinct memories, olfactory performance is also a measure of episodic memory.

Olfactory test can be performed under a level of stress. Here, a psychological stress can be applied in the form of a count-down clock, flashing light, buzzer or alike. Responses and response time of test without stress and under stress offer a greater level sensitivity for assessment of cognitive and olfactory functions, that may not be possible in a clinical setting.

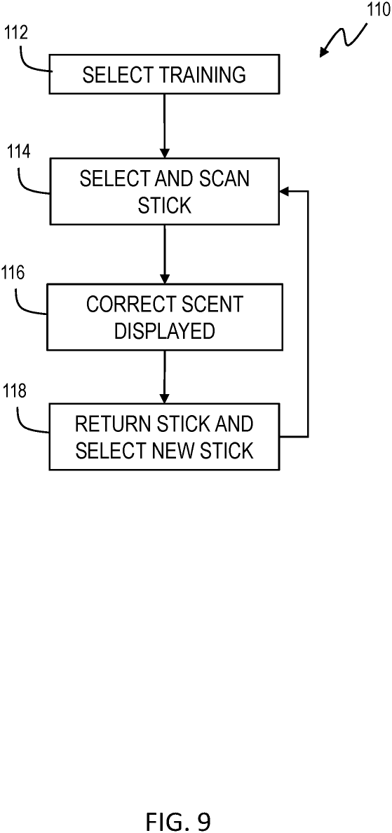

Referring to FIG. 9, software application 50 may also be used to perform olfactory training exercises 110 that are best performed after an olfactory assessment. If a user is experiencing a loss, a reduction, or a distortion of smell, olfactory training may be performed to re-train the olfactory senses to try to help regain original function. For olfactory training, a user may select olfactory training exercises in step 112, where the pattern of the questions and prompts are similar to those described above, but after scanning first indica 30 in step 114, the correct smell will be indicated in step 116. The user may then take as much time as needed to familiarize with the smell before requesting software application 50 to proceed to the next scent in step 118. This test can be stopped at any time. As an example, training may proceed as describe above, with the user launching software application 50 in a quiet place with no distractions and scanning third indicia 48 to configure mobile application 50 to the particular rack 40. The user can then select inhaler stick 14 from rack 40, smell inhaler stick 14, scan inhaler stick 14, and determine whether the smelled scent was correct. This process may be repeated for one or more additional inhaler sticks 14 in rack 40.

The olfactory test may be adapted to any cultural or regional population with odors familiar to the population.

Software application 50 may additionally be programmed to perform additional testing unrelated to olfactory functionality but related to conditions for which olfactory deterioration is associated. For example, software application 50 may perform a digit-symbol substitution test that is a well-established neuropsychological test sensitive to brain damage, dementia, age and depression. In this test, numerical digits are substituted for certain symbols. Referring to FIG. 9, the look-up table 200 for all the digits and their respective symbols may always displayed on the screen of a smart device 202 having a front facing camera 204 running software application. Symbols may be shapes with straight lines, angles, intersections, circles, and combinations thereof.

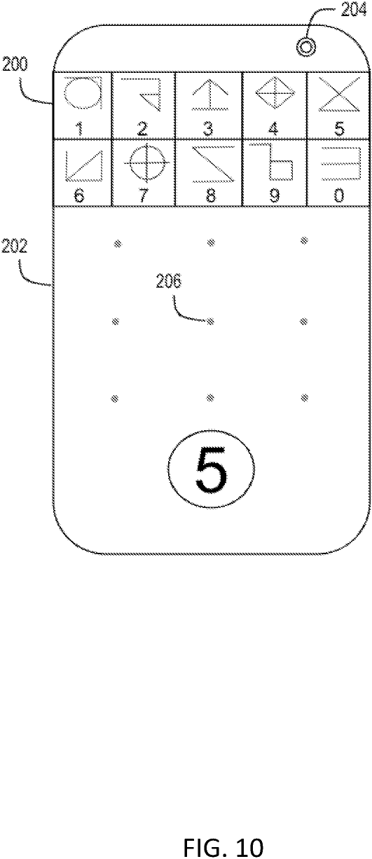

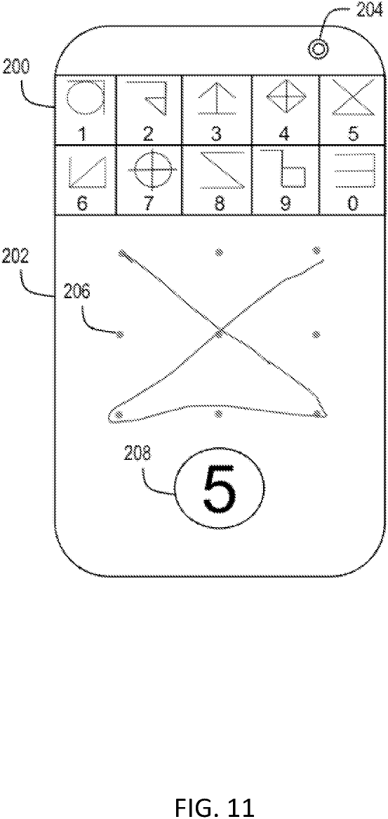

The test begins with a few practice trials, where a particular digit is displayed in location 208, and the user has to use look up table 200 to identify and then draw the symbol corresponding to the digit, as show in in FIG. 11. The digit changes after every response so that the user has to look up the corresponding symbol for this digit and draw it out in the grid. A 3×3 grid of dots 206 may be used to help guide responses. For example, a user uses his or her finger to draw the symbol that corresponds to the digit displayed in location 208 (shown as number 5 in FIGS. 10 and 11). The user has to move their eyes up and down the screen a couple of times to respond, which can be noted and recorded by camera 204. Oculomotor abnormalities often encountered in diseases including Parkinson’s disease, Gaucher disease, Neimann-Pick disease, Huntington disease, may be screened for using such a mechanism. Loss of olfaction is also observed in such conditions.

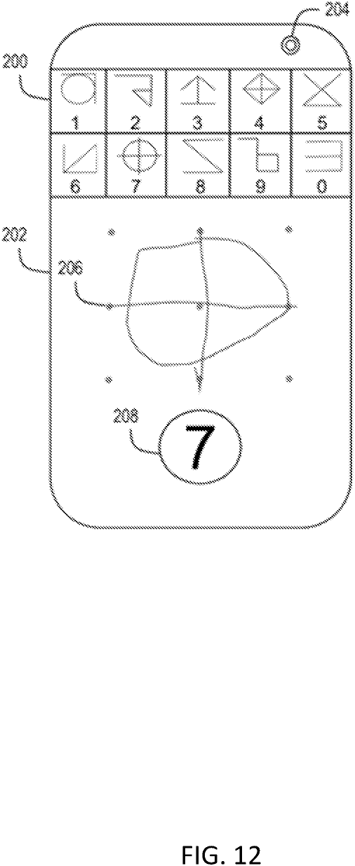

Referring to FIG. 12, the use of symbols with circles has relevance to a clock test. For example, the ability to draw a circle properly (or not) has a correlation to which part of the brain may be affected in cognitive impairment. Existing digit-symbol substitution tests do not employ a circle, and thus cannot do any brain damage mapping. By including some symbols having circular elements, such as the symbol corresponding to the digit seven in location 208, the present invention can provide information about the location of brain impairment.

After three tries, the program may shift to timed tests where a predetermined number of tests are performed. Meta-data is collected during the tests, such as eye movements captured by front camera 204, response times (or average response time), patterns in drawing straight lines/edges/angles/ circles, smoothness of lines between the dots in the grid, roundedness of circles, degree of roundedness in drawing angles, and other applicable patterns. A library of symbols that can be drawn using the 3×3 grid is created. There are four sections in the test session (three rounds of copying task and one round of recall task). Each test session has a unique combination of digits and symbols. This is done to not allow any prior memory. AI-based image processing algorithms may be applied to compare the reference image displayed in the screen with the image created by the user. The user needs to first perform three rounds of copy function where the digits for which the corresponding symbol needs to be copied. In the first round the dots in the 3×3 grid appears dark, in the next round the grid appears fainter and in the third round the grid is invisible, where the user must copy on an empty canvas. The traces offer information on visuo-perceptual response, fine motor skills, attention. After three rounds of copying (a total of copying thirty symbols), the task changes to a recall task, where the symbols disappear. The performance in the recall section of the routine provides information about user’s non-verbal memory. In a sense this part of the test is related to the Rey-Osterrieth complex Figure (ROCF) test.

A variation of the Verbal Learning Test may be incorporated in the smart device. This test assesses verbal memory of the user. In this test, the smart device speaker reads out twenty words (from a library of all 3- 6-lettered from the English language dictionary). After an adjustable interval or delay time (immediate, 5 minutes or 20 minutes) the user is asked to recall the words that were read out using the smart device. The words may be displayed on the screen and be a pool of words that were read and not read through the speaker. The user has to identify the words that was read from such a list. This test assesses a person’s verbal memory. The test variation to the verbal learning test may be applied to any language. In this manner other tests that tests different cognitive domains may also be developed as applications on a smart device including Wisconsin Card sorting test for abstract thinking,

Statistical analysis, artificial intelligence, machine learning, deep learning algorithms may then be applied to the data for image processing, weighing of difficulty levels and scoring and other analytical processes, including the identification of any correlations with smell test data such as that described above. Integration of performance parameters of various cognitive domains (verbal and non-verbal memory, executive function, processing speed, fine motor abilities, attention, abstract thinking), and olfactory responses (olfactory identification score, olfactory discrimination and threshold identification score, smell test scan-to-scan response time as an indicator of executive function, odor specific response, olfactory intra-test consistency, olfactory response under stress) is performed using statistical, artificial intelligence, machine learning, deep learning algorithms to determine threshold values for normal and disease conditions. Disease conditions may include but not limited to Alzheimer’s disease, Parkinson’s disease, amnestic and non-amnestic mild cognitive impairment (MCI), Alzheimer’s disease due to MCI, dementia, multiple sclerosis, traumatic brain injury (TBI), Huntington disease, epilepsy, neuromuscular diseases, attention deficit disorders, brain tumors, olfactory neuroblastoma, nasal polyps.

What is claimed is:

1. An olfactory testing platform, comprising:
a kit containing a plurality of scented objects, each of which includes a first indicia that is computer readable, and a second indicia that is human readable, wherein each of the scented objects provides a scent that is different than at least one other scented object; and
a computer application configured to run on a smart device having a camera, where the computer application is programmed to identify each scented object in the kit using the first indicia and to prompt a user to select a detected scent from a plurality of options in response to presentation of the first indicia to the camera of the smart device.

2. The platform of claim 1, wherein the first indicia and the second indicia are positioned on opposing sides of each of the plurality of scented objects.

3. The platform of claim 2, wherein the kit includes a rack having a plurality of receptacles for receiving the plurality of scented objects and a cover that can be positioned in covering relation to the rack to enclose the plurality of scented objects.

4. The platform of claim 3, wherein at least one of the rack and the cover includes a third indicia containing information about the plurality of scented objects.

5. The platform of claim 4, wherein the computer application is programmed to prompt the user to scan each scented object in the kit using the first indicia.

6. The platform of claim 4, wherein the computer application is programmed to prompt the user to select more than one of the plurality of scented objects and identify the scent of each of the more than one of the plurality of scented objects.

7. The platform of claim 4, wherein the computer application is programmed to prompt the user to select at least two of the plurality of scented objects and to discriminate between the at least two of the plurality of scented objects according to a strength of the scent associated with each of the at least two of the plurality of scented objects.

8. The platform of claim 4, wherein the computer application is programmed to prompt the user to scan one of the plurality of scented objects and to display the scent associated with that scented object.

9. The platform of claim 1, wherein the computer application is programmed to display a digit corresponding to one of a series of symbols in a table and to allow the user to draw the one of the series of symbols corresponding to the digit.

10. The platform of claim 9, wherein the computer application is programmed to record eye movements of the user when the user draws the one of the series of symbols corresponding to the digit.

11. The platform of claim 1, wherein the plurality of scented objects are selected from the group consisting of inhaler sticks, scented cards, scented scratch cards, scented absorbent material, and combinations thereof.

12. A method of performing olfactory testing, comprising the steps of:
providing a plurality of scented objects, each of which includes a first indicia that is computer readable, and a second indicia that is human readable, wherein each of the scented objects provides a scent that is different than at least one other inhaler stick; and
using a computer application running on a smart device having a camera to identify each inhaler stick upon presentation of the first indicia to the camera and to prompt a user to select a detected scent from a plurality of options after presentation of the first indicia to the camera.

13. The method of claim 12, further comprising the step of using the computer application to prompt the user to scan each scented object using the first indicia.

14. The method of claim 12, further comprising the step of using the computer application is programmed to prompt the user to select more than one of the plurality of scented objects and identify the scent of each of the more than one of the plurality of scented objects.

15. The method of claim 12, further comprising the step of using the computer application to prompt the user to select at least two of the plurality of scented objects and to discriminate between the at least two of the plurality of scented objects according to a strength of the scent associated with each of the at least two of the plurality of scented objects.

16. The method of claim 12, further comprising the step of using the computer application to prompt the user to scan one of the plurality of scented objects and to display the scent associated with that scented object .

17. The method of claim 12, further comprising the step of using the computer application to display a digit corresponding to one of a series of symbols in a table and to allow the user to draw the one of the series of symbols corresponding to the digit.

18. The method of claim 17, further comprising the step of using the computer application to record the eye movements of the user when the user draws the one of the series of symbols corresponding to the digit.

19. The method of claim 18, wherein the plurality of scented objects are selected from the group consisting of inhaler sticks, scented cards, scented scratch cards, scented absorbent material, and combinations thereof.
